(19) United States
(12) Patent Application Publication
(10) Pub. No.: US 2022/0028480 A1
(43) Pub. Date: Jan. 27, 2022

(54) PREDICTING AFFINITY USING STRUCTURAL AND PHYSICAL MODELING

(71) Applicant: University of Notre Dame du Lac, South Bend, IN (US)

(72) Inventors: Brian Baker, South Bend, IN (US); Tim Riley, South Bend, IN (US)

(21) Appl. No.: 17/312,107

(22) PCT Filed: Dec. 6, 2019
(86) PCT No.: PCT/US2019/064988

§ 371 Date: Jun. 9, 2021

Related U.S. Application Data
(60) Provisional application No. 62/777,670, filed on Dec. 10, 2018.

(51) Int. Cl. G16B 15/30; G16B 40/20

(52) U.S. Cl. CPC G16B 15/30; G16B 40/20

(57) ABSTRACT

Described are methods for predicting affinity of a candidate molecule for a second molecule. The method comprises obtaining a three-dimensional candidate structural representation of the candidate molecule bound to a second molecule; obtaining a plurality of candidate measurements, wherein each candidate measurement is associated with at least one feature of the candidate structural representation; and predicting, with an electronic processor, the affinity of the candidate molecule for the second molecule, wherein the electronic processor is configured to predict the affinity of the candidate molecule for the second molecule based upon the plurality of candidate measurements. The candidate molecule may be a peptide, such as a neoantigen, a viral peptide, a non-mutated self peptide, or a post-translationally modified peptide. The second molecule may be an antigen presenting molecule, such as a class I MHC molecule or a class II MHC molecule.

### CROSS-REFERENCE TO RELATED APPLICATIONS

This claims priority to U.S. Provisional Patent Application No. 62/777,670, filed on Dec. 10, 2018, the entire contents of which are fully incorporated herein by reference.

### STATEMENT OF GOVERNMENT INTEREST

This invention was made with government support under grant R35GM118166 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 11,697 bytes ASCII (Text) file named “18-072-092012-9119-WO01-SEQ-LIST_ST25.txt,” created on Dec. 5, 2019.

### TECHNICAL FIELD

The present disclosure relates to methods for predicting affinity of molecules using structural and physical modeling. In particular, the methods disclosed herein may be used to predict affinity of peptides for antigen presenting molecules.

### BACKGROUND

Successful therapeutic vaccination relying on peptide antigens presented to T cells of the immune system is a longstanding goal for cancer immunotherapy. DNA sequencing and advances in immunoinformatics have led to the identification of neoantigens incorporating nonsynonymous mutations that differentiate tumors from healthy tissues. Following sequencing of tumor material, potential neoantigens have been identified via bioinformatic approaches that predict processing and presentation by MHC proteins, and more recently, mass spectrometry. However, effective means to predict affinity of potential neoantigens for antigen presenting molecules, such as MHC proteins, are needed.

### SUMMARY

Disclosed herein are methods for predicting affinity of a candidate molecule for a second molecule. The method comprises obtaining a three-dimensional candidate structural representation of the candidate molecule bound to a second molecule; obtaining a plurality of candidate measurements, wherein each candidate measurement is associated with at least one feature of the candidate structural representation; and predicting, with an electronic processor, the affinity of the candidate molecule for the second molecule, wherein the electronic processor is configured to predict the affinity of the candidate molecule for the second molecule based upon the plurality of candidate measurements.

Other aspects of the disclosure will become apparent by consideration of the detailed description and accompanying drawings.

BRIEF DESCRIPTIONS OF THE DRAWINGS

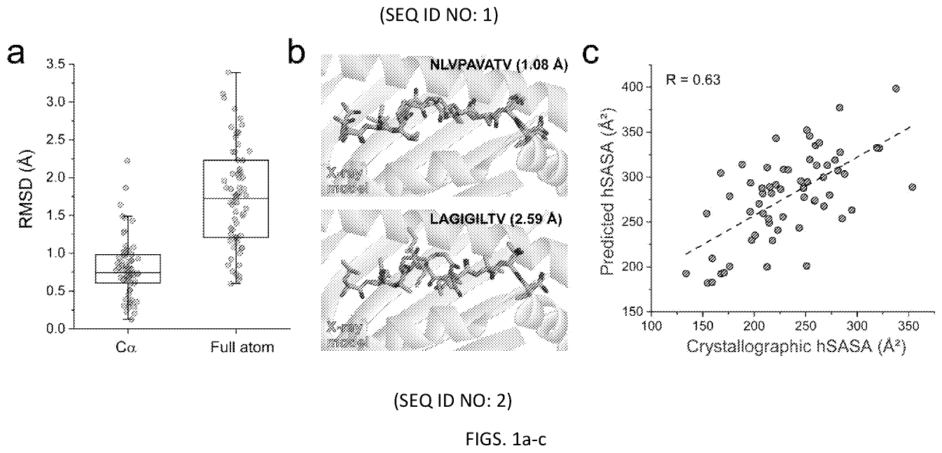

FIGS. 1a-c show rapid structural modeling for peptide/HLA-A2 complexes. FIG. 1a is a graph showing modeling performance for 62 structures, showing RMSD for modeled vs. crystallized peptides in a box and whisker plot. The left shows RMSD calculations for α carbons only; the right shows all peptide atoms. Boxes illustrate the 1^{st} and 3^{rd} quartiles, with a horizontal line at the median and a red star at the mean. Whiskers show 1.5 of the interquartile range. FIG. 1b shows structural images of representative models and their corresponding structures. The top shows the model of NLVPAVATV (SEQ ID NO: 1), which superimposes on the crystal structure with a full atom RMSD of 1.08 Å. The bottom shows the model of LAGIGILTV (SEQ ID NO: 2), which superimposes on the structure with a full atom RMSD of 2.59 Å. For the latter case, the leucine at position 1 forces the nonameric peptide to bind in a register-shifted decameric configuration, with the p1 leucine in the B-pocket. FIG. 1c is a graph showing correlation between exposed peptide hydrophobic surface area in the models vs. the crystallographic structures. The two sets of data correlate with an R value of 0.63.

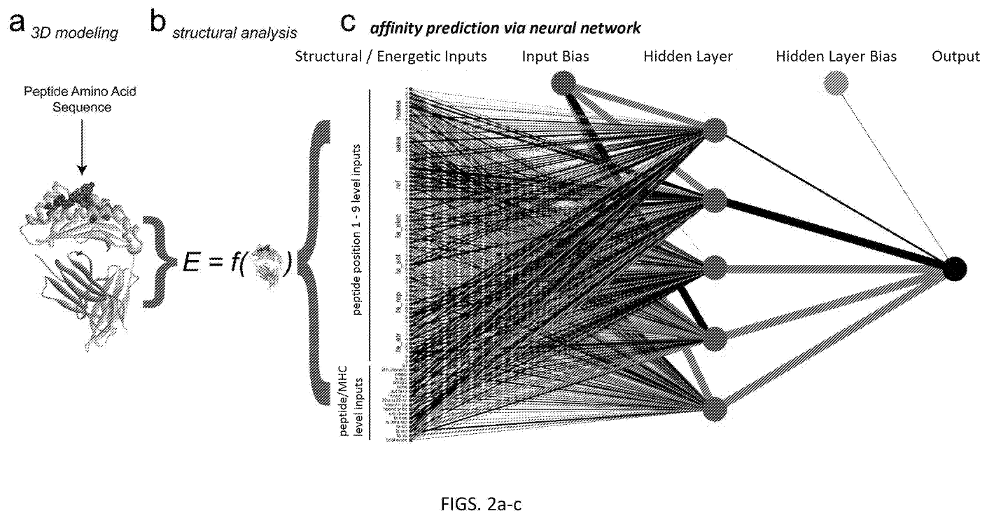

FIGS. 2a-c show the process and architecture of the structure-based affinity neural network. FIG. 2a shows the process begins with a peptide sequence, which is used to generate a model of the peptide/HLA-A2 three-dimensional structure using Rosetta. FIG. 2b shows analysis of the modeled structure yields energetic and topographical information, which are used as inputs for the structure-based affinity neural network (SBAN). FIG. 2c shows SBAN architecture, with 81 structure-derived inputs shown on the left (seven for each peptide position, 18 for the overall complex). A single hidden layer is present with five hidden neurons, along with two constant bias nodes. Black lines give positive weights, grey lines negative weights, with line width indicating weight magnitude.

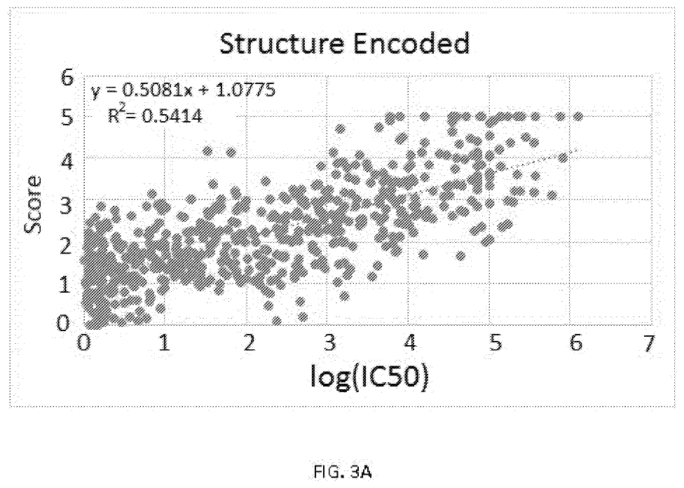

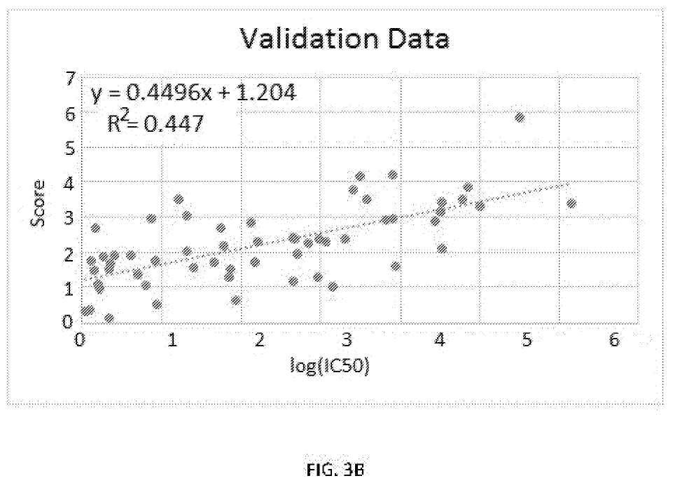

FIGS. 3A-B show performance of the structure-based affinity neural network in categorizing peptide affinity for HLA-A2. FIG. 3A is a graph showing performance of SBAN following a nested 5-fold cross validation procedure in evaluating the training data In evaluating the training data of 596 peptides, SBAN reliably predicted the experimentally determined log IC50 values previously reported (R²=0.5414). FIG. 3B is a plot showing SBAN's performance against an independent dataset of 57 nonameric peptides not used in training, indicating a robust prediction procedure (R²=0.447).

### DETAILED DESCRIPTION

Disclosed herein are methods for predicting affinity of a candidate molecule for a second molecule.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Affinity” and “binding affinity” as used interchangeably herein refers to the strength of the binding interaction between a first molecule and a second molecule. For example, affinity may refer to the strength of the binding interaction between a candidate molecule and a second molecule, or between a reference molecule and a second molecule.

### 2. Methods for Predicting Affinity

Disclosed herein are methods for predicting affinity of a candidate molecule for a second molecule. The methods described herein explicitly contemplate predicting the affinity of one candidate molecule or multiple candidate molecules for a second molecule. The methods comprise obtaining a three-dimensional candidate structural representation of the candidate molecule bound to a second molecule. The three-dimensional candidate structural representation may be generated. For example, the three-dimensional candidate structural representation may be generated using any suitable software known in the art. Alternatively, the three-dimensional candidate structural representation may be obtained from any suitable source, such as a database.

The method further comprises obtaining a plurality of candidate measurements. Each candidate measurement is associated with at least one feature of the candidate structural representation. For example, the method may comprise obtaining a plurality of candidate measurements selected from the group consisting of solvent accessible surface areas, solvation energies, hydrophobicity, electrostatic interactions, and van der Waals interactions. These measurements are listed as examples only and are not intended in any way to be limiting. Other suitable measurements may be used in addition or alternatively to these example measurements. For example, other suitable measurements are provided in Table 1.

The method further comprises predicting, with an electronic processor, the affinity of the candidate molecule for the second molecule. The electronic processor may be a microprocessor, an application-specific integrated circuit (ASIC), or other suitable electronic device. The electronic processor executes computer-readable instructions (“software”). The software may include firmware, one or more applications, program data, filters, rules, one or more program modules, and other executable instructions. For example, the software may include instructions and associated data for performing a set of functions including the methods described herein. The electronic processor may be configured to predict the affinity of the candidate molecule for the second molecule based upon the plurality of candidate measurements.

The electronic processor may be further configured to predict the affinity of the candidate molecule based upon a plurality of reference measurements. Each reference measurement may be associated with at least one feature of one or more reference structural representations. Each reference structural representation is a three-dimensional representation of a reference molecule bound to the second molecule. Each reference measurement may be selected from the group consisting of solvent accessible surface areas, solvation energies, hydrophobicity, electrostatic interactions, and van der Waals interactions. These measurements are listed as examples only and are not intended in any way to be limiting. Other suitable measurements may be used in addition or alternatively to these example measurements. For example, other suitable measurements are provided in Table 1.

Each reference molecule may have a known affinity for the second molecule. In some embodiments, the electronic processor is further configured to predict the affinity of the candidate molecule based upon the known affinity of each reference molecule for the second molecule. Suitable measures of affinity include, for example, the equilibrium dissociation constant (K_{d}), the half maximal inhibitory concentration (IC₅₀), or the melting temperature of the bi-molecular complex (Tₘ). For example, the electronic processor may be configured to predict the K_{d} of the candidate molecule for the second molecule and each reference molecule may have a known K_{d} for the second molecule. Alternatively, the electronic processor may be configured to predict the IC₅₀ of the candidate molecule and each reference molecule may have a known IC₅₀. As another alternative, the electronic processor may be configured to predict the Tₘ of the bi-molecular complex (i.e, the melting temperature of the candidate molecule when bound to the second molecule) and each reference molecule may have a known Tₘ when bound to the second molecule.

The electronic processor may be configured to predict the affinity of the candidate molecule for the second molecule using a machine-learned model trained to predict the affinity of the candidate molecule for the second molecule using the plurality of reference measurements. Machine learning generally refers to the ability of a computer program to learn without being explicitly programmed. In some embodiments, a computer program is configured to construct a model (one or more algorithms) based on example inputs. Machine learning involves presenting a computer program with example inputs and their desired (for example, actual) outputs. The computer program is configured to learn a general rule (a model) that maps the inputs to the outputs. The computer program may be configured to perform machine learning using various types of methods and mechanisms. For example, the computer program may perform machine learning using decision tree learning, association rule learning, artificial neural networks, inductive logic programming, support vector machines, clustering, Bayesian networks, reinforcement learning, representation learning, similarity and metric learning, sparse dictionary learning, or genetic algorithms.

The second molecule may be any desired molecule. For example, the second molecule may be an antigen presenting molecule. For example, the antigen presenting molecule may be an MEW molecule. In some embodiments, the antigen presenting molecule is a class I MHC molecule or a class II MHC molecule. For example, the antigen presenting molecule may be HLA-A2.

The candidate molecule may be any desired molecule. In some embodiments, the candidate molecule may be a peptide. For example, the candidate molecule may be a neoantigen, a viral peptide, a non-mutated self peptide, or a post-translationally modified peptide.

### 3. EXAMPLES

The following Examples are offered as illustrative as a partial scope and particular embodiments of the disclosure and are not meant to be limiting of the scope of the disclosure.

### Example 1

### Methods

Structural modeling of HLA A2 presented peptides: Structural modeling of peptide/HLA-A2 complexes was performed with PyRosetta using the Talaris2014 energy function. The desired peptide sequence was computationally introduced into HLA-A2, using PDB ID 3QFD (2^{nd} molecule in the asymmetric unit) as a template for nonamers and 1JF1 as a template for decamers. This was followed by 50 Monte Carlo-based simulated annealing sidechain and peptide backbone minimization steps using the LoopMover_Refine_CCD protocol, generating 20 independent decoys per peptide. The large number of resulting packing operations introduced some minor variability when scoring the models. Therefore, the unweighted score terms for the three lowest scoring trajectories were averaged and used for neural network inputs.

Collection of data sets: The structural database for evaluating modeling strategies consisted of high resolution (<3.0 Å) nonameric or decameric peptide/HLA-A2 structures within the PDB. Structures in this dataset were selected for strong electron density as determined by visual inspection using COOT for calculating 2Fₒ-F_{c} density maps. The final database contained 62 structures presenting different peptide epitopes (56 nonamers and 6 decamers). For structures with multiple molecules in the asymmetric unit, RMSDs of modeled peptides were calculated to all molecules and the lowest RMSD value was reported.

Artificial neural network training: The neural network training set contained 596 HLA-A201 restricted peptides collected from Kim et al., BMC Bioinformatics (2014) 15:241 for an equivalent IC50 distribution ranging from 0.01 nm to 1,250,000 nM.

Two-layer feed-forward networks were trained with the scaled conjugate gradient back-propagation training tool in Matlab 2017b. Training and evaluation of neural network architectures was performed using a nested five-fold cross-validation procedure. The peptides in the training dataset were split into five sets of training, validation, and test data. Using the reported log(IC50) values to classify each peptide, the training data were used to perform feed-forward and back propagation. The validation set defined the stopping criteria for the network training, and the test set evaluated performance via Correlation Coefficient. Sets were rotated to ensure each was used in training, validation, and testing. The average R of all the test sets, reported as an indicator of overall performance, was 0.65.

The neural network architecture used was a conventional feed-forward network with an input layer containing 81 neurons, one hidden layer with 5 neurons, and a single neuron output layer. The neurons in the input layer describe structural and structure-derived energetic-features of the 9 amino acids in the peptide sequence, with each amino acid represented by up to 11 neurons. The remaining 18 neurons describe global structural and structure-derived energetic features of the entire peptide/HLA-A2 complex. The structural and energetic features were those that comprise the Talaris2014 energy function or derived from the structure as listed in Table 1.

TABLE 1
Structural and structure-derived terms used for training
the structure based affinity network. Energetic terms
are those that comprise the Talaris2014 energy function

| | Description |
|---|---|
| Global energetic terms | |
| describing entire peptide/ | |
| MHC complex | |
| total_score | Total Talaris 2014 total energy |
| fa_atr | Total Lennard-Jones attractive |
| fa_rep | Total Lennard-Jones repulsive |
| fa_sol | Total Lazaridus-Karplus solvation energy |
| fa_intra_rep | Total Lennard-Jones repulsive between |
| | atoms of same residue |
| fa_elec | Total Coulombic electrostatic potential |
| pro_close | Total proline ring closure energy |
| hbond_sr_bb | Total backbone-backbone hydrogen bond |
| | energy, close in structure |
| hbond_lr_bb | Total backbone-backbone hydrogen bond |
| | energy, distant in structure |
| hbond_bb_sc | Total sidechain-backbone hydrogen bond |
| | energy |
| hbond_sc | Total sidechain-sidechain hydrogen bond |
| | energy |
| dslf_fa13 | Total disulfide geometry potential |
| rama | Total ramachandran preference energy |
| omega | Total omega dihedral energy in the |
| | backbone |
| fa_dun | Total internal energy of sidechain rotamers |
| | as derived from Dunbrack's statistics |
| p_aa_pp | Total probability of amino acid at phipsi |
| yhh_planarity | Total torsional potential for Tyrosine |
| ref | Total of reference energies for each amino |
| | acid |
| Energetic terms at the level of | |
| each peptide amino acid | |
| fa_atr | Lennard-Jones attractive (beween position |
| | atoms and every other atom of pMHC) |
| fa_rep | Lennard-Jones repulsive (beween position |
| | atoms and every other atom of pMHC) |
| fa_sol | Lazaridus-Karplus solvation energy for |
| | position |
| fa_intra_rep (excluded after | Lennard-Jones repulsive between atoms of |
| cross-validation) | same residue |
| fa_elec | Coulombic electrostatic potential (beween |
| | position and every other atom of pMHC) |
| rama (excluded after cross- | Ramachandran preferences |
| validation) | |
| fa_dun (excluded after cross- | Internal energy of sidechain rotamers as |
| validation) | derived from Dunbrack's statistics |
| p_aa_pp (excluded after | Probability of amino acid at phipsi |
| cross-validation) | |
| ref | Amino acid reference energy for position |
| Additional amino acid level | |
| terms (structure dervied, non- | |
| energetic) | |
| sasa | Solvent accessible surface area |
| hsasa | Hydrophobic solvent accessible surface |
| | area |

### Example 2

### Development and Performance of a Rapid pMHC Modeling Strategy

To develop a rapid structural modeling strategy, an extensive list of peptide/WIC structures within the PDB were compiled. Analysis was restricted to high resolution HLA-A2 structures with good electron density throughout the length of the peptide. To emphasize structural differences emerging from amino acid changes, the database was further narrowed by pairing each peptide/HLA-A2 complex with at least one other in which the peptide differed by only a single amino acid, either as a substitution or transposition. The final database contained 62 structures presenting distinct peptide epitopes (56 nonamers and 6 decamers) (Table 2).

TABLE 2
Structures utilized in benchmarking
structural modeling

| | | Talaris | | |
|---|---|---|---|---|
| PDB | | 2014 | FA RMSD | Ca RMSD |
| Entry | Sequence | energy ^{a} | (Å) ^{b} | (Å) ^{c} |
| 3qfd | AAGIGILTV | −488.09 | 0.69 | 0.45 |
| | (SEQ ID NO: 3) | | | |
| 1jht | ALGIGILTV | −488.69 | 0.76 | 0.35 |
| | (SEQ ID NO: 4) | | | |
| 1b0g | ALWGFFPVL | −446.86 | 3.11 | 0.96 |
| | (SEQ ID NO: 5) | | | |
| 1i7u | ALWGFVPVL | −460.77 | 3.39 | 1.86 |
| | (SEQ ID NO: 6) | | | |
| 1i7t | ALWGVFPVL | −464.75 | 2.91 | 0.98 |
| | (SEQ ID NO: 7) | | | |
| 3mrj | CINGMCWTV | −476.58 | 2.00 | 0.66 |
| | (SEQ ID NO: 8) | | | |
| 3mrg | CINGVCWTV | −471.70 | 1.68 | 0.89 |
| | (SEQ ID NO: 9) | | | |
| 3mrl | CINGVVWTV | −473.66 | 1.31 | 0.83 |
| | (SEQ ID NO: 10) | | | |
| 3mrh | CISGVCWTV | −477.16 | 1.67 | 0.78 |
| | (SEQ ID NO: 11) | | | |
| 2gt9 | EAAGIGILTV | −507.03 | 0.72 | 0.20 |
| | (SEQ ID NO: 12) | | | |
| 2gtw | LAGIGILTV | −484.17 | 2.59 | 2.23 |
| | (SEQ ID NO: 2) | | | |
| 1jfl | ELAGIGILTV | −508.13 | 0.65 | 0.13 |
| | (SEQ ID NO: 13) | | | |
| 4jfo | ALAGIGILTV | −512.17 | 0.60 | 0.29 |
| | (SEQ ID NO: 14) | | | |
| 3mro | ELAGWGILTV | −505.14 | 0.92 | 0.29 |
| | (SEQ ID NO: 15) | | | |
| 5hhp | GILEFVFTL | −467.52 | 1.45 | 0.31 |
| | (SEQ ID NO: 16) | | | |
| 2vll | GILGFVFTL | −467.42 | 2.35 | 0.95 |
| | (SEQ ID NO: 17) | | | |
| 5hhn | GILGLVFTL | −468.41 | 1.64 | 0.72 |
| | (SEQ ID NO: 18) | | | |
| 5hhq | GIWGFVFTL | −436.19 | 1.21 | 0.52 |
| | (SEQ ID NO: 19) | | | |
| 3mrf | GLCPLVAML | −470.61 | 1.50 | 0.68 |
| | (SEQ ID NO: 20) | | | |
| 3mre | GLCTLVAML | −470.29 | 1.77 | 1.05 |
| | (SEQ ID NO: 21) | | | |
| 2x4u | ILKEPVHGV | −280.78 | 0.84 | 0.35 |
| | (SEQ ID NO: 22) | | | |
| 1ilf | FLKEPVHGV | −289.95 | 0.84 | 0.23 |
| | (SEQ ID NO: 23) | | | |
| 1ily | YLKEPVHGV | −295.73 | 1.00 | 0.39 |
| | (SEQ ID NO: 24) | | | |
| 1eez | ILSALVGIL | −472.34 | 1.85 | 1.04 |
| | (SEQ ID NO: 25) | | | |
| 1eey | ILSALVGIV | −470.09 | 1.25 | 0.68 |
| | (SEQ ID NO: 26) | | | |
| 1tvh | IMDQVPFSV | −478.66 | 1.67 | 0.80 |
| | (SEQ ID NO: 27) | | | |
| 1tvb | ITDQVPFSV | −480.64 | 1.55 | 0.77 |
| | (SEQ ID NO: 28) | | | |
| 3v5h | KVAEIVHFL | −469.46 | 1.79 | 0.84 |
| | (SEQ ID NO: 29) | | | |
| 3v5d | KVAELVHFL | −434.03 | 1.59 | 0.36 |
| | (SEQ ID NO: 30) | | | |
| 3v5k | KVAELVWFL | −473.24 | 2.53 | 0.88 |
| | (SEQ ID NO: 31) | | | |
| 3pwl | LGYGFVNYI | −435.88 | 3.06 | 1.29 |
| | (SEQ ID NO: 32) | | | |
| 3pwn | LLYGFVNYI | −431.36 | 2.70 | 0.91 |
| | (SEQ ID NO: 33) | | | |
| 3pwj | LLYGFVNYV | −464.42 | 2.60 | 1.43 |
| | (SEQ ID NO: 34) | | | |
| 2git | LLFGKPVYV | −433.12 | 2.34 | 0.90 |
| | (SEQ ID NO: 35) | | | |
| 1im3 | LLFGYPVYV | −383.66 | 2.44 | 0.69 |
| | (SEQ ID NO: 36) | | | |
| 3mrc | NLVPMCATV | −476.86 | 2.67 | 1.47 |
| | (SEQ ID NO: 37) | | | |
| 3mrd | NLVPMGATV | −472.02 | 1.20 | 0.93 |
| | (SEQ ID NO: 38) | | | |
| 3gsw | NLVPMVAAV | −469.65 | 1.52 | 0.89 |
| | (SEQ ID NO: 39) | | | |
| 3gso | NLVPMVATV | −474.97 | 1.14 | 0.38 |
| | (SEQ ID NO: 40) | | | |
| 3gsx | NLVPMVAVV | −473.99 | 1.07 | 0.51 |
| | (SEQ ID NO: 41) | | | |
| 3mrb | NLVPMVHTV | −477.22 | 1.23 | 0.65 |
| | (SEQ ID NO: 42) | | | |
| 3gsv | NLVPQVATV | −475.90 | 1.39 | 0.75 |
| | (SEQ ID NO: 43) | | | |
| 3gsq | NLVPSVATV | −478.06 | 1.16 | 0.65 |
| | (SEQ ID NO: 44) | | | |
| 3gsu | NLVPTVATV | −474.65 | 1.21 | 0.74 |
| | (SEQ ID NO: 45) | | | |
| 3gsr | NLVPVVATV | −465.23 | 1.07 | 0.61 |
| | (SEQ ID NO: 46) | | | |
| 3mr9 | NLVPAVATV | −476.83 | 1.08 | 0.70 |
| | (SEQ ID NO: 1) | | | |
| 3mgo | RLYQNPTTYI | −276.50 | 1.96 | 0.66 |
| | (SEQ ID NO: 47) | | | |
| 3mgt | KLYQNPTTYI | −269.34 | 2.78 | 1.64 |
| | (SEQ ID NO: 48) | | | |
| 1s8d | SLANTVATL | −475.50 | 1.33 | 0.71 |
| | (SEQ ID NO: 49) | | | |
| 2v2x | SLFNTVATL | −475.95 | 2.60 | 1.49 |
| | (SEQ ID NO: 50) | | | |
| 1s9x | SLLMWITQA | −470.59 | 2.08 | 1.10 |
| | (SEQ ID NO: 51) | | | |
| 1s9w | SLLMWITQC | −470.56 | 2.34 | 1.27 |
| | (SEQ ID NO: 52) | | | |
| 3k1a | SLLMWITQL | −459.05 | 1.87 | 0.99 |
| | (SEQ ID NO: 53) | | | |
| 1s9y | SLLMWITQS | −469.11 | 2.07 | 1.08 |
| | (SEQ ID NO: 54) | | | |
| 1t1x | SLYLTVATL | −464.44 | 2.00 | 0.60 |
| | (SEQ ID NO: 55) | | | |
| 1t20 | SLYNTIATL | −469.84 | 2.11 | 0.71 |
| | (SEQ ID NO: 56) | | | |
| 2v2w | SLYNTVATL | −461.86 | 2.04 | 0.63 |
| | (SEQ ID NO: 57) | | | |
| 1t1y | SLYNVVATL | −365.42 | 2.05 | 0.74 |
| | (SEQ ID NO: 58) | | | |
| 3ft3 | VLHDDLLEA | −457.06 | 1.87 | 0.74 |
| | (SEQ ID NO: 59) | | | |
| 3ft4 | VLRDDLLEA | −442.43 | 2.23 | 1.02 |
| | (SEQ ID NO: 60) | | | |
| 3myj | YMFPNAPYL | −394.92 | 2.23 | 0.81 |
| | (SEQ ID NO: 61) | | | |
| 3hpj | RMFPNAPYL | −463.57 | 1.85 | 1.00 |
| | (SEQ ID NO: 62) | | | |

^{a} Total energy of modeled peptide/HLA-A2 complex as scored by the Talaris 2014 score function in Rosetta energy units
^{b} full atom RMSD of modeled peptide to structure
^{c} a carbon RMSD of modeled peptide to structure

Modeling speed was prioritized over complexity. Nonameric and decameric peptides bound to class I MHC proteins adopt relatively conserved backbone conformations. Therefore, each complex in the database was modeled by threading the desired peptide sequence into template HLA-A2 structures, followed by Monte-Carlo-based conformational sampling and energy minimization for side chains and the peptide backbones utilizing Rosetta. This approach, which required approximately 10 minutes per model on 2016-vintage CPU hardware, predicted the experimentally determined structures with a mean peptide Cα root mean square deviation (RMSD) of 0.8 Å and full-atom RMSD of 1.8 Å (FIG. 1A; Table 2). The greatest discrepancy between modeled and actual structures was an unusual register-shifted nonameric peptide (LAGIGILTV) (SEQ ID NO: 2) which, compared to the native peptide (AAGIGILTV) (SEQ ID NO: 3), left the p1 pocket of the HLA-A2 molecule empty in the crystal structure, so the nonameric peptide adopted a decameric configuration (FIG. 1B). The rapid modeling procedure was not able to sample such dramatic conformational shifts, and thus the model of this peptide resembled more traditional nonameric peptide/MHC structures.

Other approaches to model peptides in class I MHC binding grooves have incorporated docking, molecular dynamics simulations, protein threading, or combinations of these methods. These other methods have reported Cα or full-atom RMSD values between model and experiment within the approximate range of 1-2 Å. The approach described herein thus compares favorably with or even outperforms other efforts.

Given recent attention on the role of exposed surface features in the immunogenicity of MHC-presented peptides, it was evaluated how the modeling procedure recovered peptide hydrophobic solvent accessible surface area (hSASA). After comparing models and structures, the correlation between predicted and experimental hSASA was 0.63 (FIG. 1C). The modeling procedure provides a good approximation of peptide structural properties within the binding groove of HLA-A2 and the changes that occur upon mutation.

### Example 3

### A Neural Network to Predict Affinity

Using the structural modeling procedure and the database of peptides described herein, an artificial neural network was constructed to predict the affinity of nonameric peptides for HLA-A2, relying on structural and energetic features determined from three-dimensional models as the network inputs. Accordingly, structural models of all 596 peptide/HLA-A2 complexes were generated. To describe the conformation-dependent physical properties of the peptides in the binding groove, the 18 terms in the Talaris2014 energy function commonly used for computational protein design were used to evaluate the energy of the entire peptide/HLA-A2 complex. The terms, listed in Table 1, account for features such as energies of attraction, repulsion, and solvation; energies of side chain and backbone hydrogen bonds; and energies and probabilities of side chain and backbone conformations. Nine terms from the same energy function for all nine positions in the peptide were also selected, selecting terms that emphasized atomic-level features and avoiding those descriptive of particular amino acids (e.g., tyrosine planarity). To the nine amino-acid level terms, total and hydrophobic solvent accessible surface areas were added. Overall then, 117 terms that describe each modeled peptide/HLA-A2 complex were used as network inputs. To maintain linearity, the log of each reported IC₅₀ was taken and used as categorization labels for each peptide.

In developing the neural network, a nested 5-fold cross-validation procedure that eliminated redundant terms was used. The final model consisted of the 18 terms for the entire peptide/MHC complex and seven for each amino acid in the peptide, yielding 81 terms for network inputs, with five hidden neurons and two constant bias nodes (FIG. 2; Table 1). The average cross-validated Pearson's coefficient (R) was 0.65. After training with the entire dataset, the final neural network (termed Structure Based Affinity Network, or SBAN) classified all peptides used with an R value of 0.74 (FIG. 3A).

### Example 5

### Testing Performance on an Unrelated Data Set Not Used in Training

SBAN scores positively correlated with log(IC50) measurements in the training data. To further evaluate performance, 57 additional HLA-A201 restricted peptides not used in training were inspected. These peptides comprise a real-world test of the disclosed model. Results are shown in FIG. 2B. Although in general performance for all models was weaker with this dataset, SBAN again positively correlated with experimentally determined log(IC50) values with an R value of 0.67 (FIG. 3B).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A method for predicting affinity of a candidate molecule for a second molecule, the method comprising:
a. Obtaining a three-dimensional candidate structural representation of the candidate molecule bound to the second molecule;
b. Obtaining a plurality of candidate measurements, wherein each candidate measurement is associated with at least one feature of the candidate structural representation;
c. Predicting, with an electronic processor, the affinity of the candidate molecule for the second molecule, wherein the electronic processor is configured to predict the affinity of the candidate molecule for the second molecule based upon the plurality of candidate measurements.

2. The method of claim 1, wherein the electronic processor is further configured to predict the affinity of the candidate molecule for the second molecule based upon a plurality of reference measurements,
wherein each reference measurement is associated with at least one feature of one or more reference structural representations,
wherein each reference structural representation is a three-dimensional representation of a reference molecule bound to the second molecule,
wherein each reference molecule has a known affinity for the second molecule.

3. The method of claim 2, wherein the electronic processor is configured to predict the equilibrium dissociation constant (K_{d}) of the candidate peptide for the second molecule, and wherein each reference molecule has a known K_{d} for the second molecule.

4. The method of claim 2, wherein the electronic processor is configured to predict the half maximal inhibitory concentration (IC₅₀) of the candidate molecule, and wherein each reference molecule has a known IC₅₀.

5. The method of claim 2, wherein the electronic processor is configured to predict the melting temperature (Tₘ) of the candidate molecule when bound to the second molecule, and wherein each reference molecule has a known Tₘ when bound to the second molecule.

6. The method of claim 2, wherein the electronic processor is configured to predict the affinity of the candidate molecule for the second molecule using a machine-learned model trained to predict affinity of the candidate molecule for the second molecule using the plurality of reference measurements.

7. The method of claim 2, wherein the electronic processor is further configured to predict the affinity of the candidate molecule for the second molecule based upon the known affinity for each reference molecule for the second molecule.

8. The method of claim 1, wherein the second molecule is an antigen presenting molecule.

9. The method of claim 8, wherein the antigen presenting molecule is a class I MHC molecule or a class II MHC molecule.

10. The method of claim 9, wherein the antigen presenting molecule is HLA-A2.

11. The method of claim 1, wherein the plurality of candidate measurements and/or the plurality of reference measurements are selected from the group consisting of solvent accessible surface areas, solvation energies, hydrophobicity, electrostatic interactions, and van der Waals interactions.

12. The method of claim 1, wherein the candidate molecule is a peptide.

13. The method of claim 12, wherein the candidate molecule is a neoantigen, a viral peptide, a non-mutated self peptide, or a post-translationally modified peptide.


SEQUENCE LISTING (62 sequences)

SEQ ID NO: 1 - protein, 9 aa, Artificial Sequence
  Synthetic
NLVPAVATV

SEQ ID NO: 2 - protein, 9 aa, Artificial Sequence
  Synthetic
LAGIGILTV

SEQ ID NO: 3 - protein, 9 aa, Artificial Sequence
  Synthetic
AAGIGILTV

SEQ ID NO: 4 - protein, 9 aa, Artificial Sequence
  Synthetic
ALGIGILTV

SEQ ID NO: 5 - protein, 9 aa, Artificial Sequence
  Synthetic
ALWGFFPVL

SEQ ID NO: 6 - protein, 9 aa, Artificial Sequence
  Synthetic
ALWGFVPVL

SEQ ID NO: 7 - protein, 9 aa, Artificial Sequence
  Synthetic
ALWGVFPVL

SEQ ID NO: 8 - protein, 9 aa, Artificial Sequence
  Synthetic
CINGMCWTV

SEQ ID NO: 9 - protein, 9 aa, Artificial Sequence
  Synthetic
CINGVCWTV

SEQ ID NO: 10 - protein, 9 aa, Artificial Sequence
  Synthetic
CINGVVWTV

SEQ ID NO: 11 - protein, 9 aa, Artificial Sequence
  Synthetic
CISGVCWTV

SEQ ID NO: 12 - protein, 10 aa, Artificial Sequence
  Synthetic
EAAGIGILTV

SEQ ID NO: 13 - protein, 10 aa, Artificial Sequence
  Synthetic
ELAGIGILTV

SEQ ID NO: 14 - protein, 10 aa, Artificial Sequence
  Synthetic
ALAGIGILTV

SEQ ID NO: 15 - protein, 10 aa, Artificial Sequence
  Synthetic
ELAGWGILTV

SEQ ID NO: 16 - protein, 9 aa, Artificial Sequence
  Synthetic
GILEFVFTL

SEQ ID NO: 17 - protein, 9 aa, Artificial Sequence
  Synthetic
GILGFVFTL

SEQ ID NO: 18 - protein, 9 aa, Artificial Sequence
  Synthetic
GILGLVFTL

SEQ ID NO: 19 - protein, 9 aa, Artificial Sequence
  Synthetic
GIWGFVFTL

SEQ ID NO: 20 - protein, 9 aa, Artificial Sequence
  Synthetic
GLCPLVAML

SEQ ID NO: 21 - protein, 9 aa, Artificial Sequence
  Synthetic
GLCTLVAML

SEQ ID NO: 22 - protein, 9 aa, Artificial Sequence
  Synthetic
ILKEPVHGV

SEQ ID NO: 23 - protein, 9 aa, Artificial Sequence
  Synthetic
FLKEPVHGV

SEQ ID NO: 24 - protein, 9 aa, Artificial Sequence
  Synthetic
YLKEPVHGV

SEQ ID NO: 25 - protein, 9 aa, Artificial Sequence
  Synthetic
ILSALVGIL

SEQ ID NO: 26 - protein, 9 aa, Artificial Sequence
  Synthetic
ILSALVGIV

SEQ ID NO: 27 - protein, 9 aa, Artificial Sequence
  Synthetic
IMDQVPFSV

SEQ ID NO: 28 - protein, 9 aa, Artificial Sequence
  Synthetic
ITDQVPFSV

SEQ ID NO: 29 - protein, 9 aa, Artificial Sequence
  Synthetic
KVAEIVHFL

SEQ ID NO: 30 - protein, 9 aa, Artificial Sequence
  Synthetic
KVAELVHFL

SEQ ID NO: 31 - protein, 9 aa, Artificial Sequence
  Synthetic
KVAELVWFL

SEQ ID NO: 32 - protein, 9 aa, Artificial Sequence
  Synthetic
LGYGFVNYI

SEQ ID NO: 33 - protein, 9 aa, Artificial Sequence
  Synthetic
LLYGFVNYI

SEQ ID NO: 34 - protein, 9 aa, Artificial Sequence
  Synthetic
LLYGFVNYV

SEQ ID NO: 35 - protein, 9 aa, Artificial Sequence
  Synthetic
LLFGKPVYV

SEQ ID NO: 36 - protein, 9 aa, Artificial Sequence
  Synthetic
LLFGYPVYV

SEQ ID NO: 37 - protein, 9 aa, Artificial Sequence
  Synthetic
NLVPMCATV

SEQ ID NO: 38 - protein, 9 aa, Artificial Sequence
  Synthetic
NLVPMGATV

SEQ ID NO: 39 - protein, 9 aa, Artificial Sequence
  Synthetic
NLVPMVAAV

SEQ ID NO: 40 - protein, 9 aa, Artificial Sequence
  Synthetic
NLVPMVATV

SEQ ID NO: 41 - protein, 9 aa, Artificial Sequence
  Synthetic
NLVPMVAVV

SEQ ID NO: 42 - protein, 9 aa, Artificial Sequence
  Synthetic
NLVPMVHTV

SEQ ID NO: 43 - protein, 9 aa, Artificial Sequence
  Synthetic
NLVPQVATV

SEQ ID NO: 44 - protein, 9 aa, Artificial Sequence
  Synthetic
NLVPSVATV

SEQ ID NO: 45 - protein, 9 aa, Artificial Sequence
  Synthetic
NLVPTVATV

SEQ ID NO: 46 - protein, 9 aa, Artificial Sequence
  Synthetic
NLVPVVATV

SEQ ID NO: 47 - protein, 10 aa, Artificial Sequence
  Synthetic
RLYQNPTTYI

SEQ ID NO: 48 - protein, 10 aa, Artificial Sequence
  Synthetic
KLYQNPTTYI

SEQ ID NO: 49 - protein, 9 aa, Artificial Sequence
  Synthetic
SLANTVATL

SEQ ID NO: 50 - protein, 9 aa, Artificial Sequence
  SLFNTVATL
SLFNTVATL

SEQ ID NO: 51 - protein, 9 aa, Artificial Sequence
  Synthetic
SLLMWITQA

SEQ ID NO: 52 - protein, 9 aa, Artificial Sequence
  Synthetic
SLLMWITQC

SEQ ID NO: 53 - protein, 9 aa, Artificial Sequence
  Synthetic
SLLMWITQL

SEQ ID NO: 54 - protein, 9 aa, Artificial Sequence
  Synthetic
SLLMWITQS

SEQ ID NO: 55 - protein, 9 aa, Artificial Sequence
  Synthetic
SLYLTVATL

SEQ ID NO: 56 - protein, 9 aa, Artificial Sequence
  Synthetic
SLYNTIATL

SEQ ID NO: 57 - protein, 9 aa, Artificial Sequence
  Synthetic
SLYNTVATL

SEQ ID NO: 58 - protein, 9 aa, Artificial Sequence
  Synthetic
SLYNVVATL

SEQ ID NO: 59 - protein, 9 aa, Artificial Sequence
  Synthetic
VLHDDLLEA

SEQ ID NO: 60 - protein, 9 aa, Artificial Sequence
  Synthetic
VLRDDLLEA

SEQ ID NO: 61 - protein, 9 aa, Artificial Sequence
  Synthetic
YMFPNAPYL

SEQ ID NO: 62 - protein, 9 aa, Artificial Sequence
  Synthetic
RMFPNAPYL